(19) United States
(12) Patent Application Publication
(10) Pub. No.: US 2003/0176809 A1
(43) Pub. Date: Sep. 18, 2003

(54) Probe cover of a tympanic thermometer and method for manufacturing the same

(76) Inventor: Kevin Lin, Hsinchu City (TW)

Correspondence Address: Keith Kline,, PRO-TECHTOR INTERNATIONAL SERVICES, 20775 Norada Court, Saratoga, CA, 95070-3018, US

(21) Appl. No.: 10/097,823

(22) Filed: Mar. 13, 2002

(51) Int. Cl. A61B 5/00

(52) U.S. Cl. 600/549

(57) ABSTRACT

The invention provides a probe cover for a tympanic thermometer. The probe cover includes a base and an infrared transparent sheath. The base is formed with an opening at a central portion thereof, and is in the form of a ring-shaped sheet. The infrared transparent sheath includes a closed surface and an open end attached to the base. A circumferential wall is formed from the closed surface to the open end. The length of the circumferential wall is smaller than that of the probe of the tympanic thermometer, and the area of the closed surface is greater than that of a front surface of the probe of the tympanic thermometer. A method for manufacturing the probe cover is also disclosed.

### BACKGROUND OF THE INVENTION

1. Field of the Invention

The present invention relates generally to a probe cover, and more particularly, to a probe cover of a tympanic thermometer and a method for manufacturing the same.

2. Description of the Related Art

Clinical thermometers are very helpful in the diagnosis of diseases. The tympanic membrane is generally considered by the medical community to be superior to oral, rectal, or underarm sites for representing the body's core temperature. The temperature of the tympanic membrane can be measured by detecting the infrared radiation from the tympanic membrane in the ear canal. In addition, the time needed for the body temperature measuring by using an infrared thermometer is short. The use of infrared thermometers therefore has become more and more prevalent.

To measure the infrared emission in the external ear canal, the probe of a tympanic thermometer has to be inserted into the external ear canal for sensing the infrared emission from the tympanic membrane for determining the temperature of the tympanic membrane. However, a tympanic thermometer that is used to determine the body temperature of different patients can cause contamination and infections. To prevent this disadvantage, the probe of a tympanic thermometer can be provided with a probe cover that is disposable after use.

Referring to FIG. 1, a probe cover of a tympanic thermometer disclosed in U.S. Pat. No. 5,163,418 to Fraden et al. includes a sheath 31 and ring-shaped base 32. The sheath 31 is formed from an infrared transparent film having a thickness of 0.001 inch. The closed end of the sheath 31 is flat and acts as a window 311 for infrared emission. The circumferential wall 312 includes a plurality of pleats. The circumferential peripheral flange is attached to the ring-shaped base 32. The base 32 is designed to fit tightly on the probe of the thermometer. When the base 32 is fitted on the probe of the thermometer, the window 311 contacts with the front inlet of the probe. The drawback of the probe cover lies in that when it is inserted in the ear canal, the pleats on the wall 312 of the sheath 31 scrape the skin of the canal and cause a patient feel uncomfortable. In addition, the sheath generates noises when it is pressed. Moreover, wrinkles appear on the window 311 when the probe cover is fitted on the probe and cause stray radiation that adversely affects the accuracy of the measured temperature.

Referring to FIG. 2, a probe cover of a tympanic thermometer disclosed in U.S. Pat. No. 5,088,834 to Howe et al. includes a frustum-shaped sheath 41 and a base 42 formed from a single piece of infrared transparent material. The dimensions of the sheath 41 are chosen to let it fit on the probe closely. The thickness of the sheath 41 is gradually from the proximal portion to the distal portion such that the window 411 has the minimal thickness of approximately 0.001 to 0.0005 inch. The base 42 circumferentially engages with the open end (proximal end) of the sheath 41 for secure the sheath 41 on the probe of the thermometer. This type of sheath has no pleats and is comfortable for use. However, one disadvantage lies in that the sheath is formed by plastically deforming the material from its proximal open end to its distal closed end. The material is plastically deformed in a significant length so that the maximum thickness change of the material is from 0.03 to 0.0005 inch and this causes difficulty in the quality control during the manufacturing process. Furthermore, because the dimensions of the sheath 41 are chosen to let it fit on the probe closely, the probe and the sheath 41 are fifted closely. Therefore, another disadvantage lies in that the heat transfer to the sheath 41 may adversely influence the measured results.

### SUMMARY OF THE INVENTION

It is therefore an object of the invention to provide a probe cover for a tympanic thermometer and a method for manufacturing the same, wherein the manufacturing processes can be simplified and a comfortable usage of the probe cover can also be provided.

Another object of the invention to provide a probe cover for a tympanic thermometer and a method for manufacturing the same, wherein the heat transfer can be reduced and a comfortable usage of the probe cover can also be provided.

To achieve the above-mentioned objects, the invention provides a probe cover for a tympanic thermometer. The probe cover includes a base and an infrared transparent sheath. The base is formed with an opening at a central portion thereof, and is in the form of a ring-shaped sheet. The infrared transparent sheath includes a closed surface and an open end attached to the base. A circumferential wall is formed from the closed surface to the open end. The length of the circumferential wall is smaller than that of the probe of the tympanic thermometer, and the area of the closed surface is greater than that of a front surface of the probe of the tympanic thermometer. A method for manufacturing the probe cover is also disclosed.

The method for manufacturing a probe of a tympanic thermometer includes the steps of:

forming a base by way of injection molding, the base being formed with an opening at a central portion thereof and in the form of a ring-shaped sheet;

cutting an infrared transparent film;

placing the base on a punch table with a puncher of a punching device penetrating through and projecting a first distance over the opening of the base, and placing the film on the puncher and the base;

pressing the film downward to bond to the base;

removing a part of the film at an outer periphery of the base; and

causing the puncher of the punching device to punch the film over a second distance.

### BRIEF DESCRIPTION OF THE DRAWINGS

These and other objects and advantages of the present invention will become apparent by reference to the following description and accompanying drawings wherein:

FIG. 1 is an elevational view showing a probe cover for an infrared thermometer disclosed in U.S. Pat. No. 5,163,418;

FIG. 2 is a pictorial view showing a probe cover for an infrared thermometer disclosed in U.S. Pat. No. 5,088,834;

FIG. 3 is a top view showing a probe cover in accordance with a preferred embodiment of the invention;

FIG. 4 is a side view showing the probe cover in accordance with the preferred embodiment of the invention;

FIG. 5 is an exploded side view showing that the probe cover of the invention is fitted with the probe of the tympanic thermometer;

FIG. 6 is a side view showing that the probe cover of the invention is fitted with the probe of the tympanic thermometer;

FIGS. 7 to 10 are schematic illustrations showing the steps for manufacturing the probe cover in accordance with the preferred embodiment of the invention; and

FIG. 11 is a cross-sectional view showing a puncher of a punching device in accordance with the preferred embodiment of the invention.

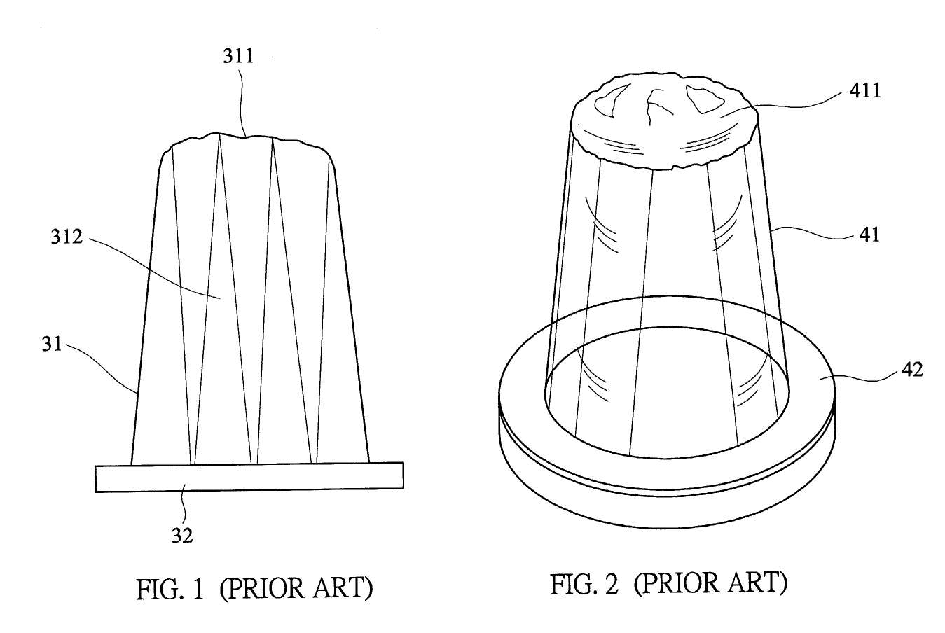

### DETAIL DESCRIPTION OF THE INVENTION

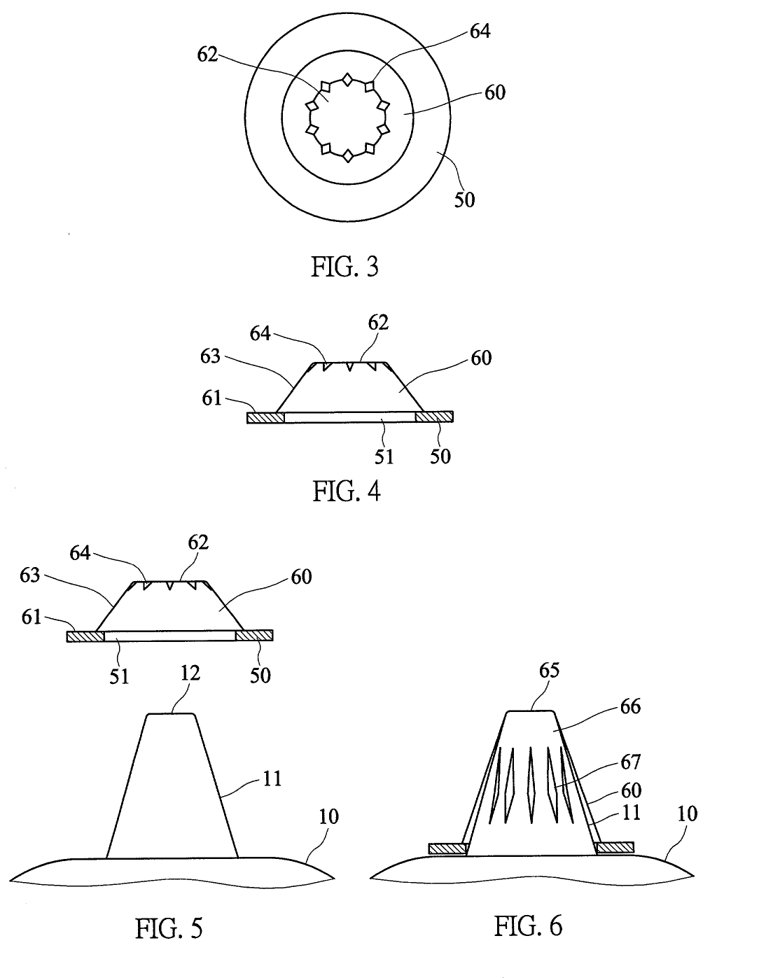

Referring to FIGS. 3 and 4, a probe cover in accordance with a preferred embodiment of the invention includes a base 50 and a sheath 60. The base 50 is a ring-shaped sheet formed with an opening 51 at the central portion thereof.

The sheath 60 is an infrared transparent sheath, and is made of a polyethylene film or a polypropylene film. The sheath 60 has an open end 61 attached to the base 50, and a closed surface 62. A circumferential wall 63 is formed from the closed surface 62 to the open end 61. The periphery of the closed surface 62 is formed with continuous pleats 64. Referring to FIG. 5, the circumferential wall 63 is shorter than the probe 11 of the tympanic thermometer 10, the cross-sectional area of the closed surface 62 is larger than that of the front surface 12 of the probe of the tympanic thermometer.

Referring to FIGS. 5 and 6, when using the above-mentioned structure, the sheath 60 is forcedly fitted in the probe 11 of the tympanic thermometer 10 to cause the sheath 60 to deform and extend a distance. Therefore, a smaller closed surface 65 is fitted with the front surface 12 of the probe. At the same time, the front section 66 of the circumferential wall has a smooth surface and is fitted with the probe 11. The middle and rear sections of the circumferential wall are formed with continuous strip pleats 67 because of the stretch of the continuous pleats 64. Accordingly, when the probe of a tympanic thermometer is inserted into the external ear canal for measuring the ear temperature, only the front section of the probe is inserted into the external ear canal. However, since the front section is covered with a smooth sheath without pleats, the sheath is free from scraping the skin of the canal to cause a patient feel uncomfortable. Furthermore, the strip pleats 67 can cause good space isolation effects between the sheath 60 and the probe 11 of the tympanic thermometer, so that heat transfer can be decreased.

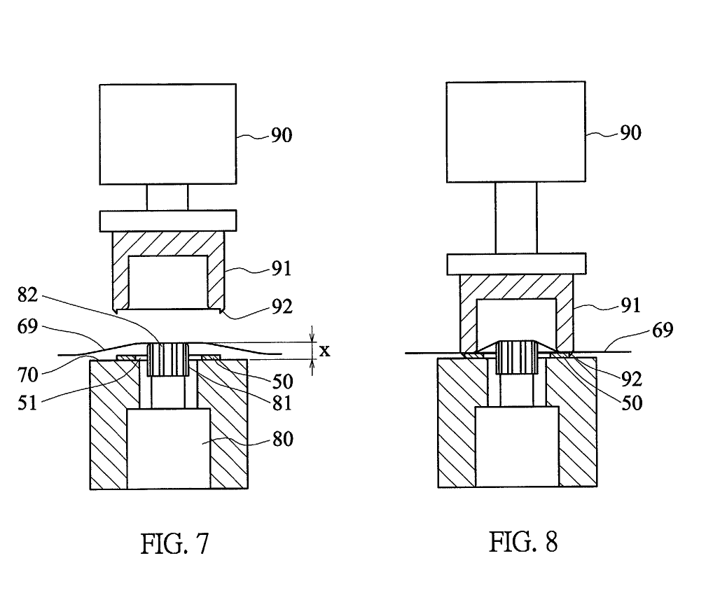

Referring to FIGS. 7 to 10, the manufacturing device in accordance with the embodiment of the invention includes a punch table 70, a punching device 80, and a heat pressing device 90. The punching device 80 is arranged below the punch table 70 and is provided with a retractable puncher 81 formed with continuous pleats 82 at its peripheral surface (FIG. 11). The cross-sectional area of the puncher 81 is larger than that of the front surface 12 of the probe of the tympanic thermometer. The heat pressing device 90 is arranged above the punch table 70 and includes a retractable pressing die 91. The pressing die 91 is a ring-shaped die having a flat bottom end with a cutting tool 92 formed at its periphery.

The method for manufacturing the sheath of the invention is described as follows.

First, a base 50, which has an opening 51 at its central portion and a ring-shaped sheet configuration, is formed by injection molding.

Next, an infrared transparent film 69 is cut.

Then, the base 50 is placed on the punch table 70 with the puncher 81 of the punching device 80 penetrating through and projecting a first distance X over the opening 51 of the base 50.

Next, the film 69 is placed on the base 50 and the puncher 81, as shown in FIG. 7.

Then, the heat pressing device 90 is activated to cause the pressing die 91 to move downward in order to press the film 69. At this time, the film 69 and the base 50 are bonded by the heat pressing processes. Simultaneously, the cutting tool 92 at the periphery of the pressing die 91 cuts out the film 69 at the periphery of the base 50, as shown in FIG. 8.

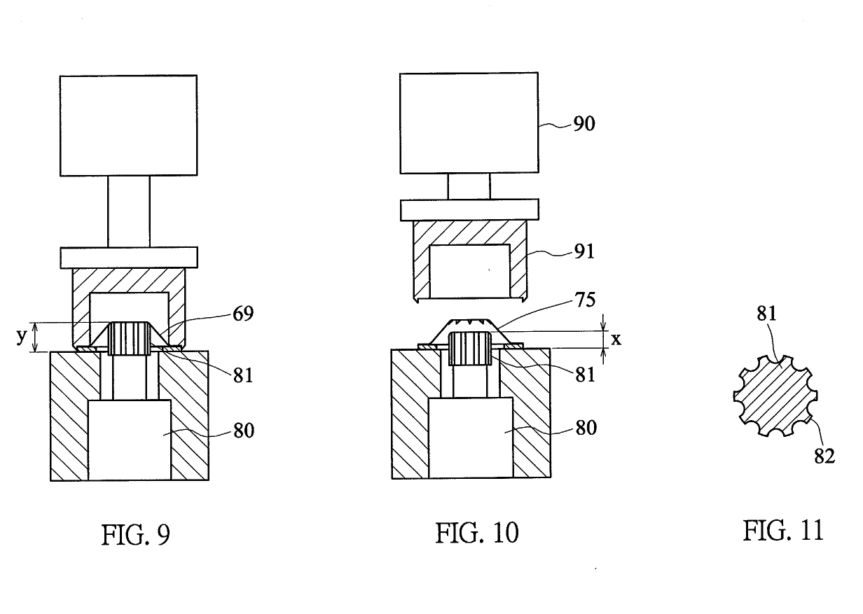

Next, the punching device 80 is activated to cause the puncher 81 to punch, from bottom to top, the film 69 over a second distance Y, as shown in FIG. 9.

Finally, the pressing die 91 of the heat pressing device 90 is returned to its initial position, and the puncher 81 is also returned to its initial position where the puncher 81 projects the first distance X over the opening 51, as shown in FIG. 10. Therefore, a facture 75 is completed and another one may be ready to be manufactured.

According to the above-mentioned description, it can be easily understood that the invention has some advantages summarized as follows.

1. Since the portion of the sheath 60 entering the external ear canal is a smooth surface, the patients never feel uncomfortable.

2. Since the middle and rear sections of the sheath are formed with continuous strip pleats 67, better spacing effects can be provided between the sheath 60 and the probe 11 of the tympanic thermometer, and heat transfer can be reduced.

3. Since the film is not fully extended, the process control is easier. Thus, no difficulty occurs in the manufacturing processes, and the qualities can be assured.

4. By punching the film to extend a second distance, the condition of whether the base 50 and the film 69 are well pressed under heat or not can be found. Thus, bad products can be found to prevent the bad products from being judged as good products. Furthermore, due to the initial extension, the sheath can be easily used when it is fitted with the probe of the tympanic thermometer and then extended.

In this invention, the area of the closed surface 62 of the sheath 60 is greater than that of the front surface 12 of the probe of the tympanic thermometer. With the area difference between the closed surface and the front surface 12, when the sheath is pushed by the probe to extend and deform, continuous strip pleats will form at the circumferential wall. In the above-mentioned embodiment, since the periphery of the puncher 81 is formed with continuous pleats, the effect of the continuous strip pleats can be more obvious.

While the invention has been described by way of an example and in terms of a preferred embodiment, it is to be understood that the invention is not limited to the disclosed embodiment. To the contrary, it is intended to cover various modifications. Therefore, the scope of the appended claims should be accorded the broadest interpretation so as to encompass all such modifications.

What is claimed is:

1. A probe cover for a tympanic thermometer, comprising:
a base formed with an opening at a central portion thereof, the base being in the form of a ring-shaped sheet; and
an infrared transparent sheath including a closed surface and an open end attached to the base, a circumferential wall being formed from the closed surface to the open end, the length of the circumferential wall is smaller than that of the probe of the tympanic thermometer, and the area of the closed surface is greater than that of a front surface of the probe of the tympanic thermometer.

2. The probe cover according to claim 1, wherein a periphery of the closed surface is formed with continuous pleats.

3. The probe cover according to claim 1, wherein the base and the sheath are bonded by heat pressing.

4. A method for manufacturing a probe of a tympanic thermometer, comprising:
forming a base by way of injection molding, the base being formed with an opening at a central portion thereof and in the form of a ring-shaped sheet;
cutting an infrared transparent film;
placing the base on a punch table with a puncher of a punching device penetrating through and projecting a first distance over the opening of the base, and placing the film on the puncher and the base;
pressing the film downward to bond to the base;
removing a part of the film at an outer periphery of the base; and
causing the puncher of the punching device to punch the film over a second distance.

5. The method according to claim 4, wherein a periphery of the puncher of the punching device is formed with continuous pleats.

6. The method according to claim 4, wherein the film and the base are bonded by a heat pressing device.

7. The method according to claim 6, wherein the heat pressing device removes a part of the film at the outer periphery of the base when the base are bonded by heat pressing.
